(19) United States
(12) Patent Application Publication
(10) Pub. No.: US 2022/0375555 A1
(43) Pub. Date: Nov. 24, 2022

(54) MEDICAL INFORMATION PROCESSING APPARATUS AND MEDICAL INFORMATION PROCESSING SYSTEM

(71) Applicant: Canon Medical Systems Corporation, Otawara-shi (JP)

(72) Inventors: Nao WARIISHI, Nasushiobara (JP); Takashi HYO, Nasushiobara (JP); Masato SHIBUYA, Otawara (JP); Hayato KONISHI, Otawara (JP)

(73) Assignee: Canon Medical Systems Corporation, Otawara-shi (JP)

(21) Appl. No.: 17/664,255

(22) Filed: May 20, 2022

(30) Foreign Application Priority Data
May 20, 2021 (JP) 2021-085475

(51) Int. Cl. G16H 10/60; G16H 30/20; G06F 3/04847; G16H 30/40; G16H 50/70

(52) U.S. Cl. CPC G16H 10/60; G16H 30/20; G06F 3/04847; G16H 30/40; G16H 50/70

(57) ABSTRACT

According to one embodiment, a medical information processing apparatus includes processing circuitry.

The processing circuitry designates one or more pieces of first clinical data among a plurality of pieces of clinical data viewed by a user. The processing circuitry specifies one or more pieces of second clinical data associated with the first clinical data among the plurality of pieces of clinical data based on a viewing history related to the plurality of pieces of clinical data. The processing circuitry outputs the first clinical data and the second clinical data.

### CROSS-REFERENCE TO RELATED APPLICATIONS

This application is based upon and claims the benefit of priority from Japanese Patent Application No. 2021-085475, filed May 20, 2021, the entire contents of which are incorporated herein by reference.

### FIELD

Embodiments described herein relate generally to a medical information processing apparatus and a medical information processing system.

### BACKGROUND

In recent years, a medical information integration viewer has been developed to integrate and enable viewing of various clinical data related to patients on a common time axis. The medical information integration viewer enables medical staff such as doctors and nurses to have a bird's-eye view of multiple pieces of clinical data on the same screen, so that the medical staff can gain a variety of insights into changes and into a relevance of each piece of data over time. Such a medical information integration viewer is desired to be a system with high convenience and operability for the user.

For example, if each piece of clinical data is displayed on a separate panel on the same screen, the user using the medical information integration viewer needs to move his or her line of sight between the panels in order to compare the data of interest. Such a display mode is not suitable because it tires the user's eyes and leads to overlooking of important data transitions. Further, if a panel is manually added to the screen so as to be a combination of clinical data that the user wants to view, it is not suitable because it leads to an increase in burden on the user. In general, the combination of clinical data that the user wants to view is determined, for example, for each user or for each patient whose clinical data is viewed. Therefore, a medical information integration viewer that specifies clinical data related to the clinical data being viewed by the user and, by extension, collectively displays the former clinical data and the latter clinical data, is desired.

### BRIEF DESCRIPTION OF THE DRAWINGS

FIG. 1 is a diagram showing a configuration example of a medical information processing system according to an embodiment.

FIG. 2 is a diagram showing a configuration example of a medical information processing apparatus according to the embodiment.

FIG. 3 is a diagram showing a first operation example of the medical information processing apparatus.

FIG. 4 is a diagram showing a first example of a viewing history.

FIG. 5 is a diagram showing a second example of a viewing history.

FIG. 6 is a diagram showing a third example of a viewing history.

FIG. 7 is a diagram showing a second operation example of the medical information processing apparatus.

FIG. 8 is a diagram showing an example of a display screen of clinical data.

FIG. 9 is a diagram showing an example of a pop-up display of related data.

FIG. 10 is a diagram showing an example of a collective display of target data and the related data.

FIG. 11 is a diagram showing a display example of a comment input window.

FIG. 12 is a diagram showing a display example of a comment reference window.

FIG. 13 is a diagram showing a display example of a layout transmission window.

### DETAILED DESCRIPTION

In general, according to one embodiment, a medical information processing apparatus includes processing circuitry.

The processing circuitry designates one or more pieces of first clinical data among a plurality of pieces of clinical data viewed by a user. The processing circuitry specifies one or more pieces of second clinical data associated with the first clinical data among the plurality of pieces of clinical data based on a viewing history related to the plurality of pieces of clinical data. The processing circuitry outputs the first clinical data and the second clinical data.

Hereinafter, a medical information processing apparatus and a medical information processing system according to an embodiment will be described with reference to the drawings. In the following embodiment, portions assigned the same reference sign perform the same operation, and redundant explanations will be omitted as appropriate.

FIG. 1 is a diagram showing a configuration example of a medical information processing system 100 according to the embodiment.

The medical information processing system 100 includes a medical information processing apparatus 1, a client terminal 2, an electronic medical record database 3, and an image database 4. Specifically, the medical information processing system 100 is a client server type system, and the medical information processing apparatus 1 performs various processing in response to a request from the client terminal 2 and returns processing results to the client terminal 2. In the medical information processing system 100, the medical information processing apparatus 1 is connected to each of the client terminal 2, the electronic medical record database 3, and the image database 4 to enable communications therebetween. The medical information processing system 100 may be, for example, an in-hospital network (LAN) constructed in a specific medical institution, or a wide area network (WAN) constructed across a plurality of medical institutions via a network. That is, the medical information processing system 100 may be a network of any scale as long as the above communication path is constructed. Further, as long as security is ensured, the connected line is not limited to the in-hospital network or the dedicated line across the medical institutions. For example, the connection may be made to a public communication line such as the Internet via a virtual private network (VPN), etc.

The medical information processing apparatus 1 is a computer adapted to process various data related to medical treatments. In the present embodiment, the medical information processing apparatus 1 functions as a server in the medical information processing system 100. The medical information processing apparatus 1 acquires various data from the electronic medical record database 3 and the image database 4 and processes them, thereby outputting various data in response to a user's request. That is, the medical information processing apparatus 1 provides a medical information integration viewer for the user to view various clinical data. The medical information processing apparatus 1 may be a workstation capable of performing high-speed processing. The medical information processing apparatus 1 may have a display unit for displaying various data and an input unit for receiving input from the user.

The client terminal 2 is a computer operated by a user such as a medical staff who uses the medical information processing apparatus 1. When there are a plurality of users, each user may have a client terminal 2. In the present embodiment, the client terminal 2 has a display unit for displaying various data transmitted from the medical information processing apparatus 1 and an input unit for receiving input to the medical information processing apparatus 1 from the user. As the display unit, any display including, for example, a cathode ray tube (CRT) display, a liquid crystal display (LCD), a plasma display, an organic electro-luminescence display (OELD), and a tablet terminal can be used. On the other hand, as the input unit, any operation component including a mouse, a keyboard, a trackball, a switch, a button, a joystick, a touch pad, and a touch panel display can be used.

The electronic medical record database 3 stores various clinical data for each patient. For example, the electronic medical record database 3 stores clinical data such as a disease name, a medical treatment phase, basic information (gender, age, height, weight, BMI, address, contact information, etc.), vital information (pulse, respiratory rate, body temperature, blood pressure, consciousness level, etc.), variance information, and a medication in association with each patient. The “variance information” includes information on clinical data of a patient who deviates from a clinical pathway. Further, the electronic medical record database 3 may be an electronic medical record system.

The image database 4 stores various medical images for each patient. For example, the image database 4 stores medical images taken by various medical image diagnostic devices (a computed radiography (CR) device, a computed tomography (CR) device, a magnetic resonance imaging (MRI) device, an ultrasound (UL) device, a radio isotope (RI) device, an endoscope device, etc. The image database 4 may be a medical image management system (PACS: Picture Archiving and Communication Systems). The medical image is an example of medical data.

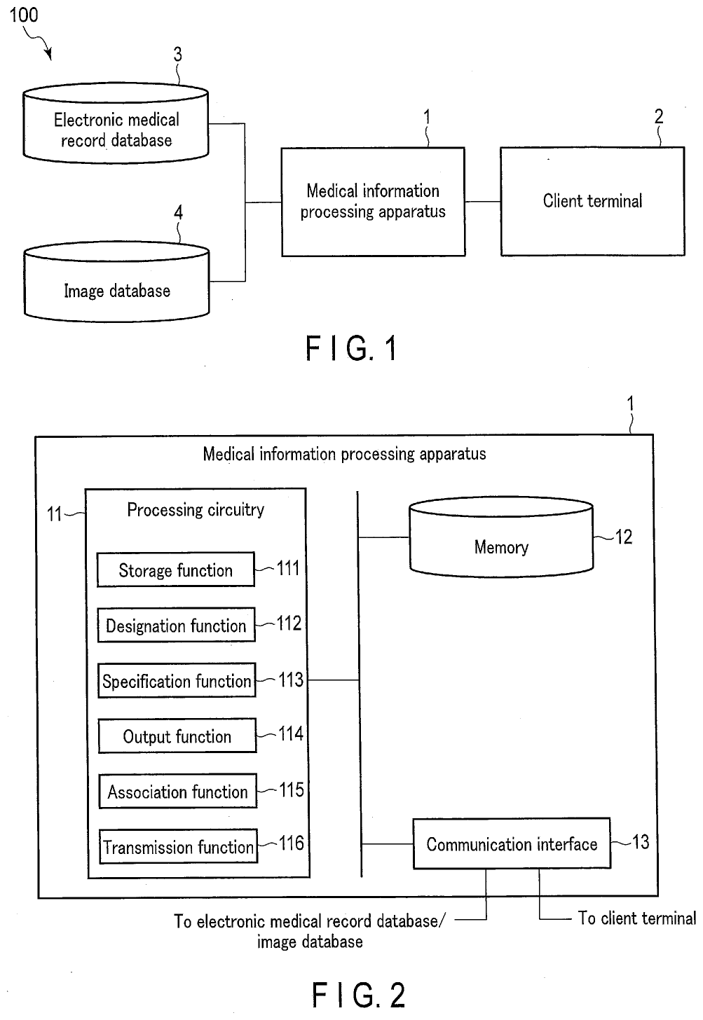

FIG. 2 is a diagram showing a configuration example of the medical information processing apparatus 1 according to the embodiment.

The medical information processing apparatus 1 includes processing circuitry 11, a memory 12, and a communication interface 13. The configurations are connected to one another via a bus which is a common signal transmission path to enable communications therebetween. Each configuration need not be realized by an individual piece of hardware. For example, at least two of the configurations may be realized by a single piece of hardware.

The processing circuitry 11 controls the medical information processing apparatus 1 to execute various operations. The processing circuitry 11 includes, as hardware, a processor such as a central processing unit (CPU), a micro processing unit (MPU), or a graphics processing unit (GPU). By executing programs developed on the memory 12 via the processor, the processing circuitry 11 realizes functions (e.g., a storage function 111, a designation function 112, a specification function 113, an output function 114, an association function 115, and a transmission function 116) corresponding to the respective programs. Each function may not be realized by the processing circuitry 11 formed of a single processor. For example, each function may be realized by the processing circuitry 11 in which a plurality of processors are combined.

The storage function 111 stores a viewing history related to a plurality of pieces of clinical data viewed by the user.

The designation function 112 designates one or more pieces of first clinical data among the plurality of pieces of clinical data.

The specification function 113 specifies one or more pieces of second clinical data related to the first clinical data among the plurality of pieces of clinical data based on the viewing history.

The output function 114 outputs the first clinical data and the second clinical data.

The association function 115 associates a comment input by a user for display data.

The transmission function 116 transmits the display data and comment to one or more other users different from the user.

The memory 12 stores data, a program, etc. used by the processing circuitry 11. The memory 12 has a semiconductor memory device such as a random access memory (RAM) as hardware. The memory 12 may be a driving device that reads and writes information to and from external storage devices, such as a magnetic disk (a floppy (registered trademark) disk, a hard disk), a magneto-optical disk (MO), an optical disk (a CD, a DVD, a Blu-ray (registered trademark)), a flash memory (a USE flash memory, a memory card, and an SSD), and a magnetic tape. A storage region of the memory 12 may be in an inner portion of the medical information processing apparatus 1 or in an external storage device. In the present embodiment, the memory 12 stores a viewing history related to a plurality of pieces of clinical data viewed by the user. The memory 12 may also store various pieces of clinical data acquired from the electronic medical record database 3 and the image database 4.

The communication interface 13 communicates various pieces of data and requests between the medical information processing apparatus 1 and the external devices (the client terminal 2, electronic medical record database 3, image database 4) connected to the apparatus to enable communications therebetween. As communication standards, DICOM (Digital Imaging and Communications in Medicine) can be used for communication related to medical image information and HL7 (Health Level 7) can be used for communication related to medical character information, for example.

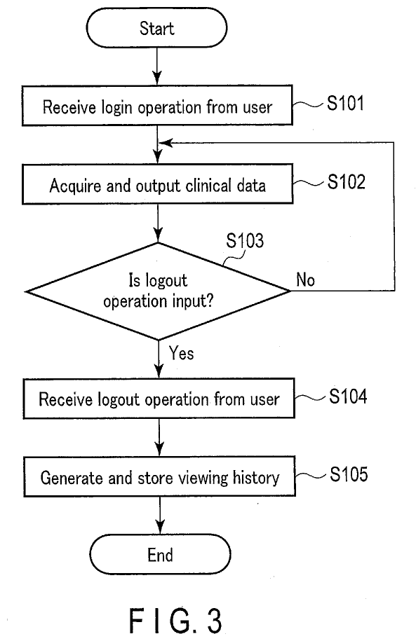

FIG. 3 is a diagram showing a first operation example of the medical information processing apparatus 1. The first operation example is an operation example in which a user uses the medical information processing apparatus 1 for the first time.

In step S101, the medical information processing apparatus 1 receives a login operation from the user. Specifically, a user who operates the client terminal 2 applies for permission to log in in order to use the medical information integration viewer provided by the medical information processing apparatus 1. For login authentication, for example, the medical information processing apparatus 1 may have a table in which a login ID and a password are associated with each user. Subsequently, if a combination of a login ID and a password input from a user who is currently requesting to log in is stored in the above table, the medical information processing apparatus 1 permits the login from that user. That is, the login operation from the user may be received by the existing authentication method.

In step S102, the medical information processing apparatus 1 acquires and outputs clinical data. Specifically, in response to a request from the user to view predetermined clinical data, the medical information processing apparatus 1 acquires the clinical data corresponding to the request from the electronic medical record database 3 and the image database 4, and performs various processing. Subsequently, the medical information processing apparatus 1 outputs the processed clinical data to the client terminal 2. At this time, the medical information processing apparatus 1 may process the acquired clinical data into display data for displaying the data in various display forms and then output the data. The above processing is repeatedly performed every time the medical information processing apparatus 1 receives a request from the user.

In step S103, the medical information processing apparatus 1 determines whether or not a logout operation from the user is input. If that operation is input (Yes in step S103), the process proceeds to step S104. If that operation is not input (No in step S103), the process returns to step S102.

In step S104, the medical information processing apparatus 1 receives the logout operation from the user. The medical information processing apparatus 1 recognizes a period from the execution of step S101 to the execution of step S104 as one login period. The login period is used as a processing unit when the medical information processing apparatus 1 generates the viewing history of a user.

In step S105, the medical information processing apparatus 1 generates and stores the viewing history of the user by the storage function 111. Specifically, the medical information processing apparatus 1 generates information on a type of each piece of clinical data viewed by the user during the login period and the time, frequency, and order in which the user views each piece of clinical data, as a viewing history. The generated viewing history is stored in the memory 12. In the present embodiment, clinical data displayed on a screen is considered to be being viewed by the user.

As described above, a viewing history may be generated by post-analyzing a viewing behavior of a user during one login period, or a viewing history may be generated in real time at each timing at which a user views each piece of clinical data. That is, a viewing history may be generated at any timing.

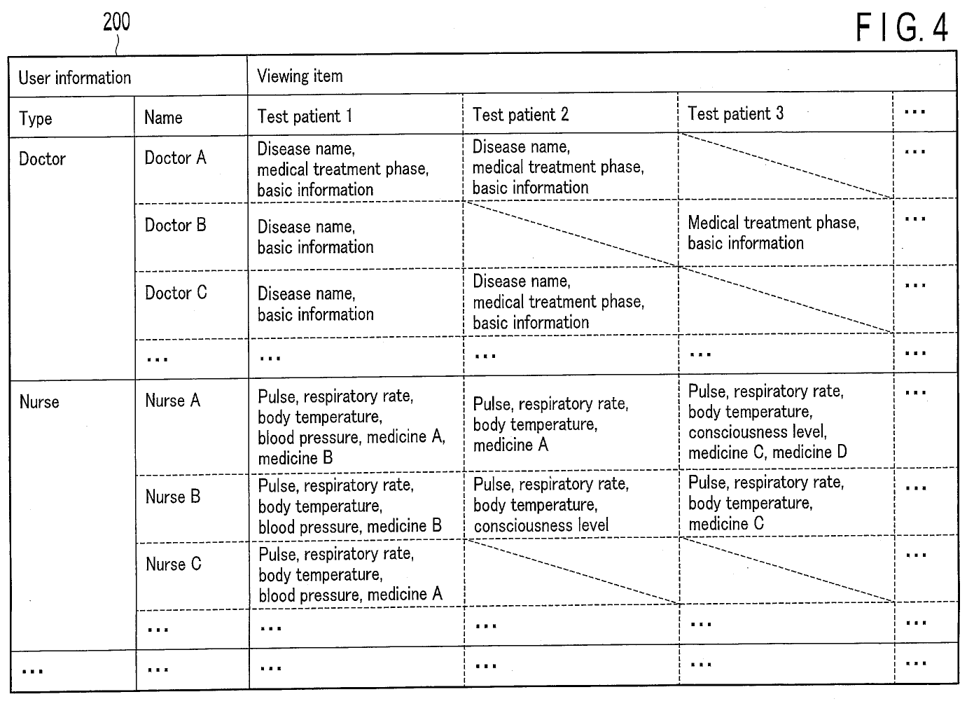

FIG. 4 is a diagram showing a first example of a viewing history.

A viewing history table 200 is a table in which a type of each piece of clinical data viewed by the user is stored. In the viewing history table 200, “type” and “name”, which are “user information”, are associated with each of a plurality of users. In addition, “viewing items”, which are clinical data viewed by each user, are associated with each patient whose clinical data is viewed.

For example, as a plurality of users whose “type” is “doctor”, the viewing items for each of “doctor A”, “doctor B”, “doctor C”, . . . are stored in association with each of “test patient 1”, “test patient 2”, “test patient 3”, . . . . Specifically, the clinical data viewed by the doctor A for the test patient 1 are “disease name”, “medical treatment phase”, and “basic information”. In the viewing history table 200, the time, frequency, and order in which each piece of clinical data is viewed may be stored in association with each piece of clinical data as a viewing time table 210 and a viewing order table 220 (to be described later). Further, in the viewing history table 200, a cell related to viewing items of a patient that a user has not yet viewed is indicated by a diagonal line.

A plurality of users whose “type” is “doctor” may be further subdivided and classified according to their respective “post” and “clinical department”. For example, a plurality of doctors may be classified according to their “post”, such as “physician”, “surgeon”, and “resident”, or their “medical care department”, such as “internal medicine”, “surgery”, and “orthopedic surgery”. A plurality of users whose “type” is “nurse”, “clinical laboratory engineer”, etc. may also be classified in the same manner as described above.

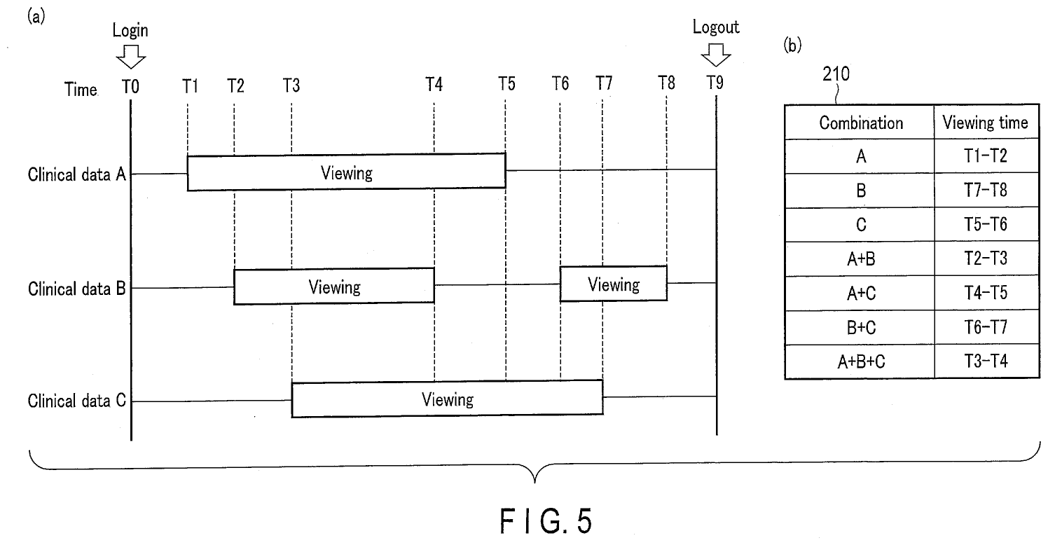

FIG. 5 is a diagram showing a second example of a viewing history. FIG. 5 is an example of a viewing history in a case in which a user can view a plurality of pieces of clinical data at the same time.

FIG. 5(a) schematically shows the time when a user views each of a plurality of pieces of clinical data (clinical data A, clinical data B, and clinical data C) on a common time axis. In the figure, time T0 is a time when the user logs in, and time T9 is a time when the user logs out. Each of times T1 to T8 is a time when the user starts or ends viewing of each piece of clinical data. It can be said that the figure shows a viewing behavior of the user during one login period (T0-T9). Hereinafter, a time zone will be expressed as “start time-end time”. For example, a time zone defined by time T1 and time T3 is expressed as “T1-T3”.

Specifically, the clinical data A is viewed in a time zone of “T1-T5”. On the other hand, the clinical data B is viewed in a time zone of “T2-T4” and a time zone of “T6-T8”. The clinical data C is viewed in a time zone of “T3-T7”. As described above, the medical information processing apparatus 1 may generate data related to a time zone in which a user views each piece of clinical data as a viewing history.

FIG. 5(b) is the viewing time table 210 related to a viewing time for each combination of the clinical data generated based on FIG. 5(a). In the viewing time table 210, for example, “combination A+B” indicates that the user views the clinical data A and B at the same time, and a viewing time in that combination is “T2-T3”. In detail, the viewing time “T2-T3” is a portion obtained by excluding a viewing time “T3-T4” of the clinical data C from the viewing time “T2-T4” in which the viewing time “T1-T5” of the clinical data A and the viewing time “T2-T4” of the clinical data B overlap. “T3-T4” may be included in the time zone at which the clinical data A and B are viewed at the same time. The medical information processing apparatus 1 calculates a viewing time for each combination of clinical data by repeating the same processing as described above, and generates the viewing time table 210.

Instead of the viewing time viewed for each combination, a viewing frequency may be calculated. In the example of FIG. 5(a), the clinical data A and B are viewed once at the same time, the clinical data A and C are viewed once at the same time, and the clinical data B and C are viewed twice at the same time. The number of times a certain piece of clinical data and another piece of clinical data are viewed at the same time is calculated based on the number of time zones at which the two pieces of clinical data are viewed at the same time. For example, since the clinical data B and C are viewed at the same time in the time zone of “T3-T4” and the time zone of “T6-T7”, it is calculated as “viewed twice at the same time”.

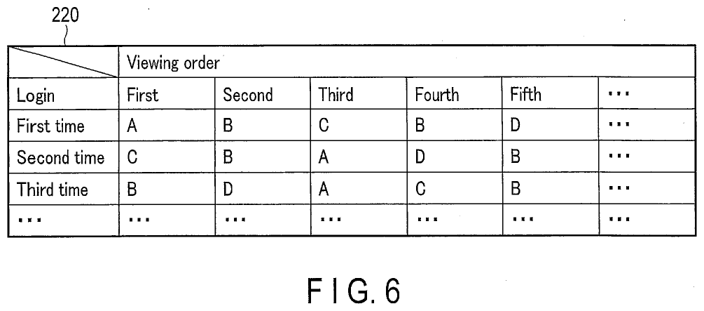

FIG. 6 is a diagram showing a third example of a viewing history. FIG. 6 is an example of a viewing history in a case in which a user can view one piece of clinical data at a time.

The viewing order table 220 shows a viewing order when the user views each piece of clinical data at the timing of each login. According to the viewing order table 220, it can be seen that in the first login, the user viewed each piece of clinical data in the order of “A→B→C→B→D . . . ” Further, in the second login, it can be seen that the user viewed each piece of clinical data in the order of “C→B→A→D→B . . . ”

Since FIGS. 5 and 6 are viewing histories generated with one login period of one user as a processing unit, the viewing time table 210 and the viewing order table 220 may be generated for each of a plurality of users. Similarly, the viewing time table 210 and the viewing order table 220 may be generated for each of a plurality of patients whose clinical data has been viewed by a user.

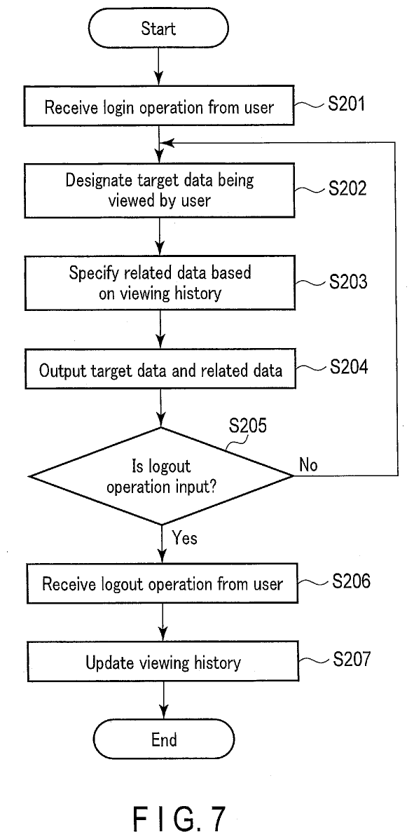

FIG. 7 is a diagram showing a second operation example of the medical information processing apparatus. The second operation example is an operation example in which the user uses the medical information processing apparatus 1 for the second time or later.

In step S201, the medical information processing apparatus 1 receives a login operation from the user. Step S201 is similar to step S101.

In step S202, the medical information processing apparatus 1 designates clinical data (also referred to as target data) being viewed by the user by the designation function 112. Specifically, the user who operates the client terminal 2 sends a request for viewing predetermined clinical data to the medical information processing apparatus 1. The medical information processing apparatus 1 designates target data corresponding to that request from a plurality of pieces of clinical data stored in the electronic medical record database 3 and the image database 4. The above process is repeatedly executed every time the user changes the target data being viewed.

In step S203, the medical information processing apparatus 1 specifies clinical data (also referred to as related data) related to the target data based on a viewing history of the user by the specification function 113. First, the medical information processing apparatus 1 specifies a user who is currently logged in and a patient whom that user is currently viewing. Next, the medical information processing apparatus 1 specifies related data based on the viewing time table 210 or the viewing order table 220 associated with a cell of the viewing history table 200 of the specified user and the specified patient.

For example, a case in which the target data is “clinical data A” in FIG. 5 is assumed. First, the medical information processing apparatus 1 specifies “combination A+B”, “combination A+C”, and “combination A+B+C” as combinations including “clinical data A” in the viewing time table 210. Next, the medical information processing apparatus 1 specifies “clinical data B” and “clinical data C” as clinical data different from “clinical data A” in each of the above combinations. In this way, the medical information processing apparatus 1 specifies “clinical data B” and “clinical data. C” as related data of “clinical data A” which is the target data.

Not limited to this, the medical information processing apparatus 1 may specify related data based on a viewing time or viewing frequency of each combination by the specification function 113. In the above example, a viewing time of “combination A+B” is “T2-T3”, a viewing time of “combination A+C” is “T4-T5”, and a viewing time of “combination A+B+C” is “T3-T4”. Next, the medical information processing apparatus 1 narrows down each combination on the condition that the combination has a viewing time or viewing frequency equal to or higher than a threshold value. At this time, since “combination A+B+C” includes “combination A+B” and “combination A+C”, its viewing time “T3-T4” is added to both of the viewing time “T2-T3” of the combination A+B and the viewing time “T4-T5” of the combination A+C. If “T2-T3”, among “T2-T3” and “T4-T5”, satisfies a viewing time equal to or greater than the threshold value, the medical information processing apparatus 1 specifies “combination A+B” as a combination that satisfies the above condition.

Subsequently, the medical information processing apparatus 1 specifies “clinical data B” as clinical data different from “clinical data A” in “combination A+B”. In this way, the medical information processing apparatus 1 specifies “clinical data B” as related data of “clinical data A” which is the target data.

The above threshold value may be set by the medical information processing apparatus 1 by default, or may be set according to an input from a user. The medical information processing apparatus 1 may specify related data on the condition that the combination has a viewing time or viewing frequency equal to or less than the threshold value.

Similarly, a case in which the target data is “clinical data A” in FIG. 6 is assumed. At this time, clinical data viewed first before and after the order in which the clinical data A was viewed is considered to have the highest relevance to the clinical data A, while clinical data viewed second before and after is considered to have the second highest relevance to the clinical data A after the clinical data viewed first before and after. In this way, clinical data viewed within ±Nth (N is a natural number) from the target data may be specified as related data of that target data. The value of “N” may be set by the medical information processing apparatus 1 by default, or may be set according to an input from a user.

In step S204, the medical information processing apparatus 1 outputs the target data and the related data by the output function 114. At this time, the medical information processing apparatus 1 processes the target data and the related data into display data for displaying them in an appropriate form on the client terminal 2, and then outputs the data. The client terminal 2 displays the output display data. As a result, the user who operates the client terminal 2 can confirm a display screen based on the display data.

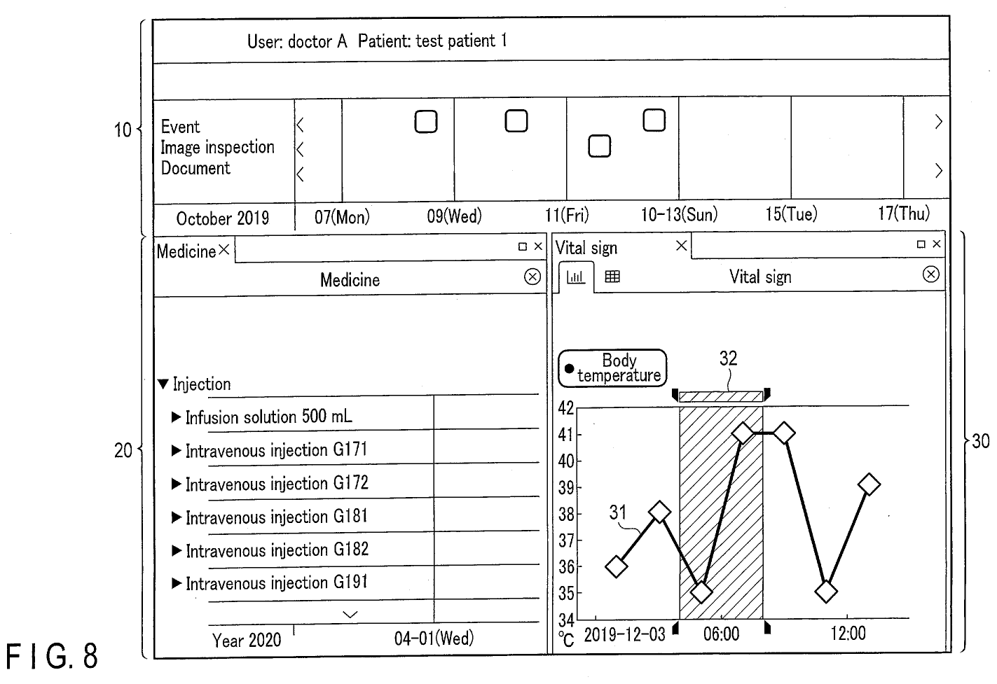

FIG. 8 is a diagram showing an example of a display screen of clinical data.

FIG. 8 is a display screen of a medical information integration viewer realized by the medical information processing apparatus 1. On this screen, a plurality of pieces of clinical data are linked on a common time axis and displayed separately on different panels. Here, it is assumed that “doctor A” in the viewing history table 200 is a logged-in user, and that “doctor A” is viewing a plurality of pieces of clinical data regarding “test patient 1”.

A panel 10 displays events, image inspections, and documents in a predetermined time zone. Times when these clinical data are present are indicated by square icons arranged on the time axis.

A panel 20 displays clinical data about medicines. Here, a list of medicines administered to “test patient 1” is displayed. As that list, for example, “infusion solution 500 mL”, “intravenous injection G171”, “intravenous injection G172”, etc. are displayed.

A panel 30 displays clinical data on vital signs. In the panel 30, as an example of vital signs, a graph 31 of a change over time of “body temperature” is displayed. Here, the user selects a desired time zone on a time axis of the graph 31 so that the time zone specified by the user is displayed as a selection range 32. Subsequently, related data regarding the selection range 32 is displayed as a pop-up 300 (to be described later). Here, it is assumed that time-series data related to a “vital sign” is target data.

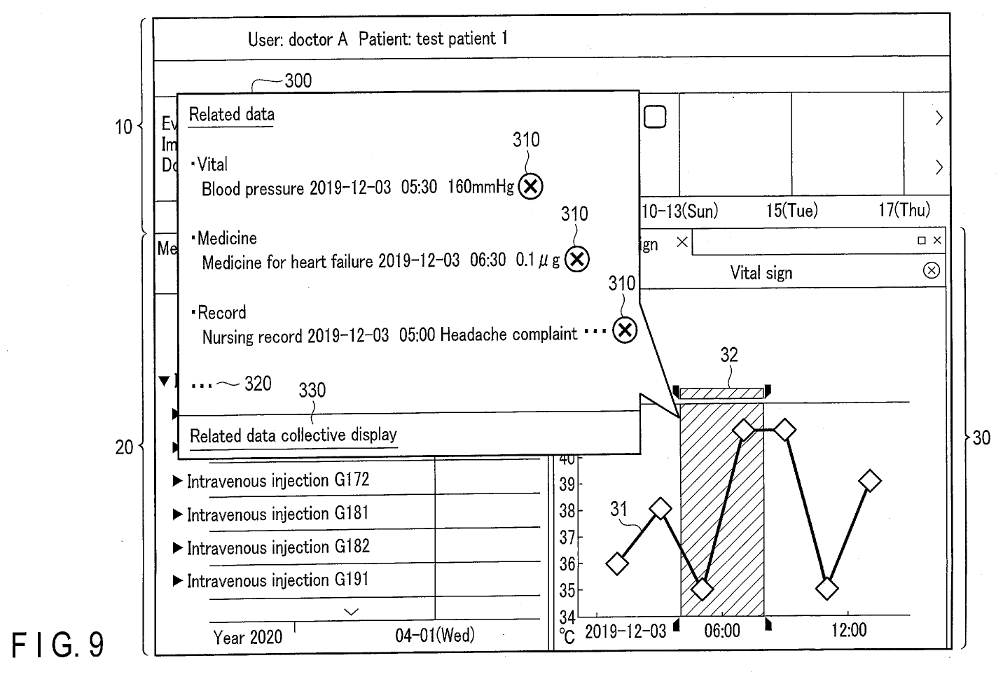

Each panel can be switched between display and non-display according to the user operation. For example, by selecting an icon of the panel 10 related to clinical data that the user wants to display, clinical data associated with the selected icon is displayed as a separate panel. Conversely, by selecting an “x button” of a panel that the user wants to hide, it is possible to close that panel. The medical information processing apparatus 1 may determine a timing at which a panel is displayed as a start of viewing clinical data on that panel by the user. Conversely, the medical information processing apparatus 1 may determine a timing at which a panel is hidden as an end of viewing clinical data on that panel by the user. FIG. 9 is a diagram showing an example of a pop-up display of related data.

The pop-up 300 indicates various related data in a time zone that coincides or approximately coincides with a time zone indicated by the selection range 32. The pop-up 300 is displayed in a form referring to the selection range 32. Specifically, in the pop-up 300, “vital: blood pressure”, “medicine: medicine for heart failure”, and “record: nursing record” are displayed as the related data. Further, as details of each of the pieces of related data, some of data values in the above time zone are displayed. A frequency of each piece of related data viewed may be displayed in association with each piece of related data. In addition, each piece of related data may be listed from the top to the bottom in order of higher viewing frequency.

Here, by the user selecting each button 310 associated with each piece of related data, it is possible to exclude the selected related data from the pop-up 300. Further, by the user selecting a button 320 expressed by a three-point lead “ . . . ”, it is possible to display other related data that is not displayed on the pop-up 300. If the button 320 is selected, the pop-up 300 enlarges and displays a display area according to the number of other hidden related data.

In addition, by the user selecting a button 330 stating “Related data collective display”, it is possible to collectively display the target data on the panel 30 and the related data on the pop-up 300 on a common time axis. At this time, a panel 40 (to be described later) is displayed on the panel 20 and the panel 30 in an overlapping manner.

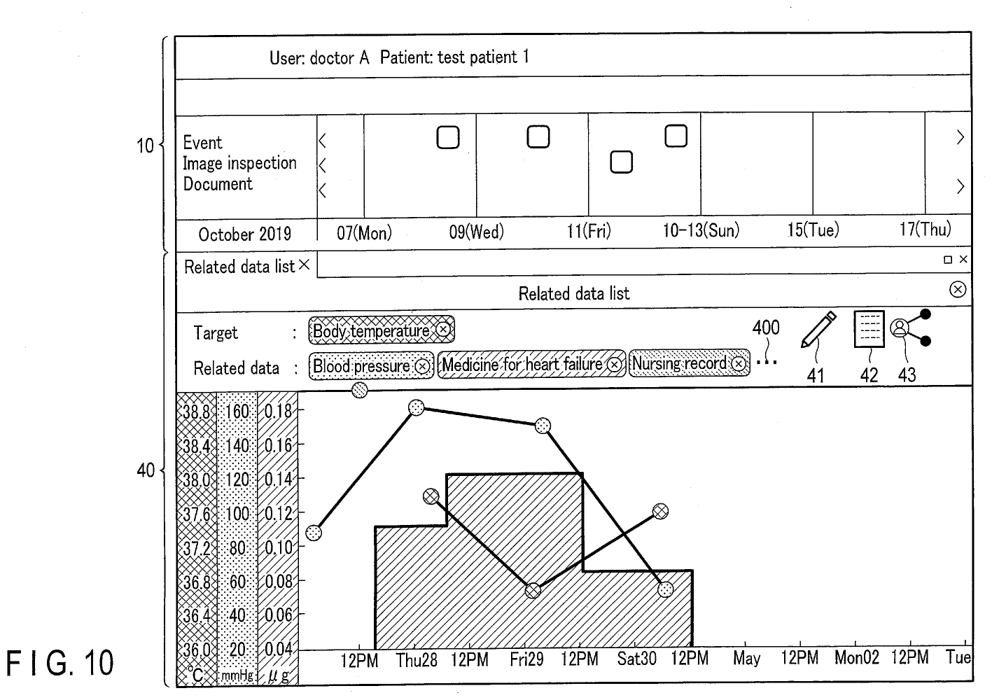

FIG. 10 is a diagram showing an example of a collective display of target data and related data.

The panel 40 shows the target data and the related data by combining them into one graph. Specifically, a temporal change of “body temperature” which is the target data (target) is displayed by a line graph. On the other hand, a temporal change of “blood pressure”, a temporal change of “medicine for heart failure”, and date and time when “nursing record” was created, which are other related data, are displayed by a line graph, a bar graph, and a plot, respectively. A display form (i.e., a line graph or a bar graph) of each graph may be set by default by the medical information processing apparatus 1, or may be set according to an input from the user. Further, a graph of the target data and a graph of the related data may be separated from each other and displayed on different pop-ups, according to the user operation.

By the user selecting a button 400 expressed by a three-point lead “ . . . ”, it is possible to further add and display a list and a graph of other related data that are not displayed on the panel 40. By the user selecting the plot for “nursing record”, a content of that selected nursing record may be displayed on a pop-up.

According to the user operation, the target data and the related data may be switched and displayed. At this time, the graph of the target data may be highlighted using a different color, a different line thickness, etc. so as to be distinguishable from the graph of the related data. Only one piece of related data or a plurality of pieces of related data may be displayed.

In addition, the medical information processing apparatus 1 may extract data showing a temporal change not satisfying a predetermined standard among the target data and the related data and issue an alert, for example, by the output function 114. For example, if a graph of “variance information” related to a respiratory rate is included as clinical data, the medical information processing apparatus 1 may detect that a change in that graph does not satisfy a standard that “respiratory rate is 12 to 30 times per minute” and issue an alert by highlighting the clinical data related to respiratory rate.

By selecting icons 41 to 43 displayed on the panel 40, the user can use various extensions according to the icons. An example of each display screen displayed when selecting each icon will be described below.

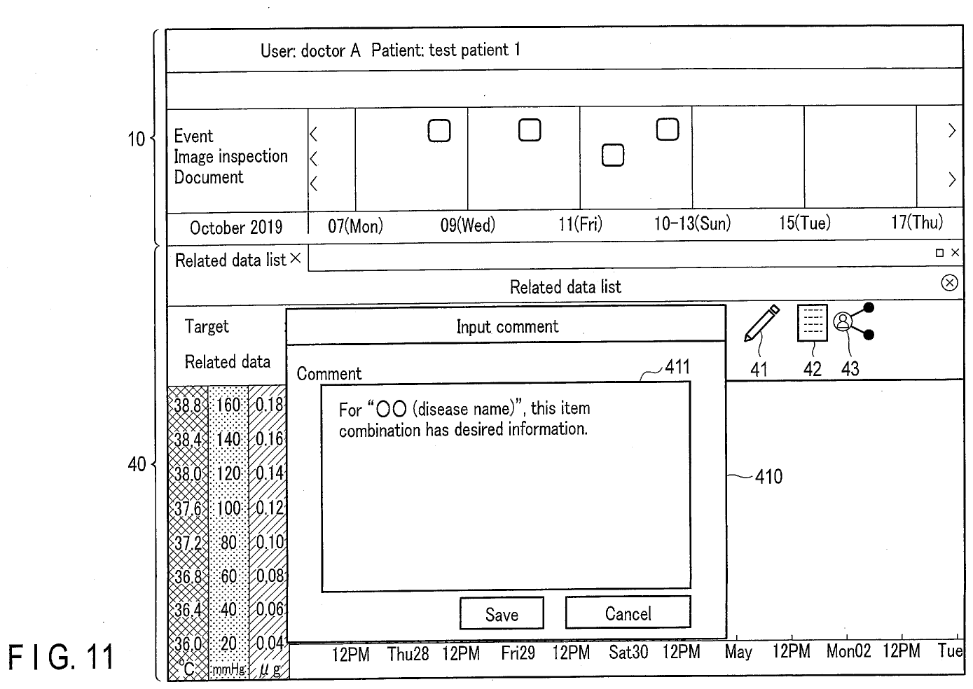

FIG. 11 is a diagram showing a display example of a comment input window.

When the user selects the icon 41, a window 410 for the user to input a comment pops up on the panel 40. The user inputs a desired comment in a box 411. After the desired comment is input, the user can save the comment in association with the panel 40 by selecting a “save” button. Thereby, the user can share the comment with other users who view the same graph.

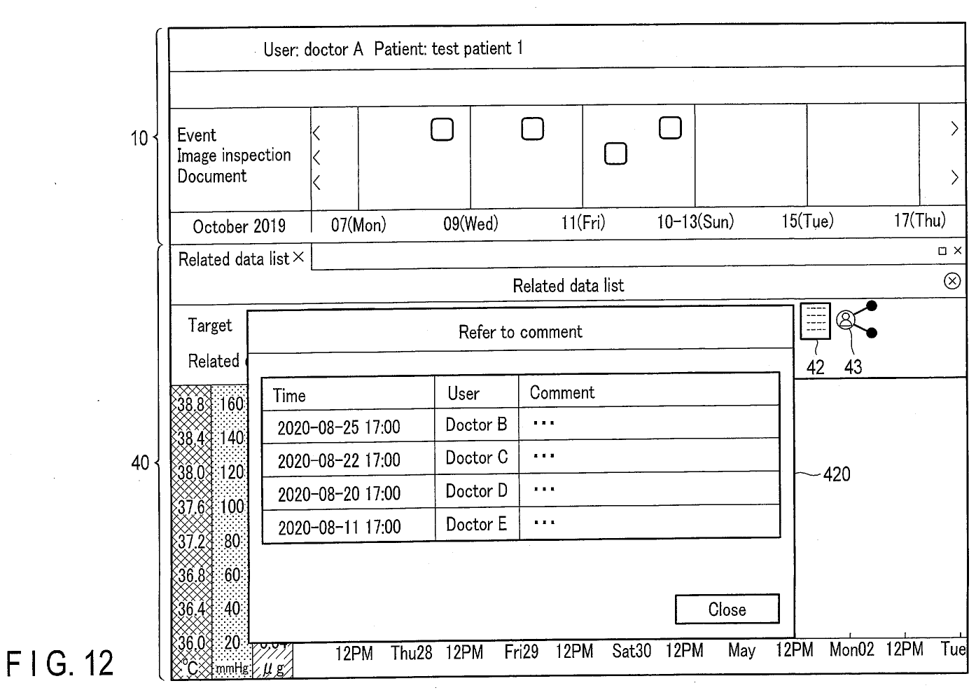

FIG. 12 is a diagram showing a display example of a comment reference window.

When the user selects the icon 42, a window 420 for the user to refer to comments input by other users pops up on the panel 40. Specifically, a list of comments created by each user (doctor B, doctor C, doctor D, and doctor E) at each time is displayed. Thereby, the user can view the comments of other users who have viewed the same graph.

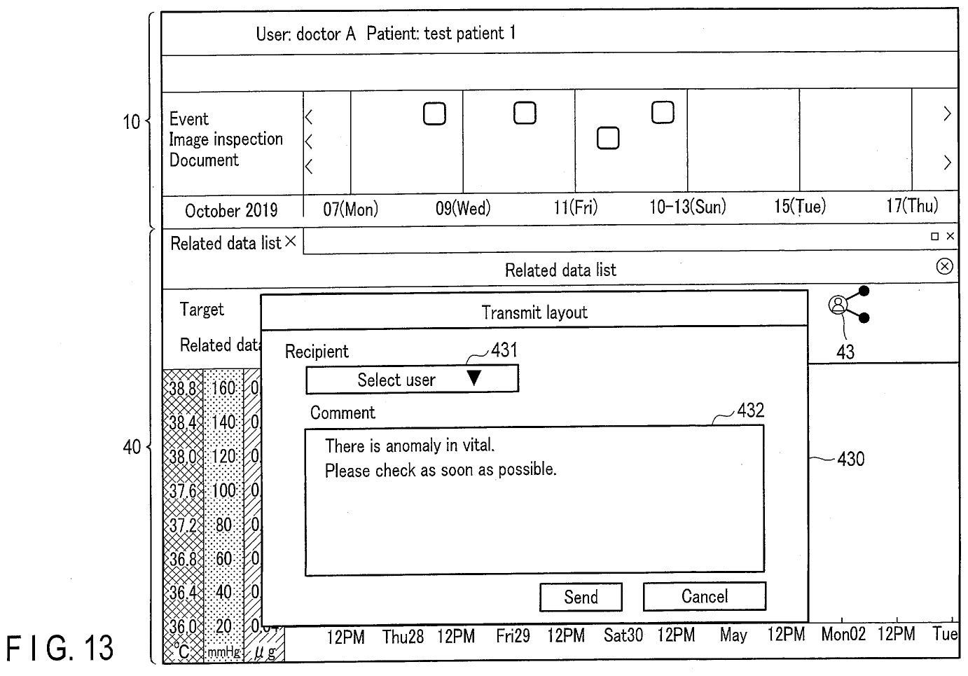

FIG. 13 is a diagram showing a display example of a layout transmission window.

When the user selects the icon 43, a window 430 for transmitting a graph of a layout being currently viewed by the user to other users pops up. In the window 430, by selecting a box 431 for specifying “recipient”, the user can select a user who is a recipient of that layout. In addition, by selecting a “send” button after inputting a desired comment in a box 432 for inputting a comment, the user can transmit a matter that the user wants the recipient of the layout to check. Thereby, for example, if a nurse finds an anomaly in clinical data of a patient being viewed, the nurse can tell the patient's doctor that the anomaly has occurred.

The explanation continues with reference back to FIG. 7.

In step S205, the medical information processing apparatus 1 determines whether or not a logout operation from the user is input. If that operation is input (Yes in step S205), the process proceeds to step S206. If that operation is not input (No in step S205), the process returns to step S202.

In step S206, the medical information processing apparatus 1 receives the logout operation from the user. Step S206 is similar to step S104.

In step S207, the medical information processing apparatus 1 updates the viewing history. Specifically, the medical information processing apparatus 1 analyzes the viewing behavior of the user during one login period from the execution of step S201 to the execution of step S206 and updates the viewing history. In other words, the medical information processing apparatus 1 adds a viewing history newly created by analyzing the viewing behavior of the user to the viewing history table 200.

Note that a time from login to logout is set as a processing unit of a viewing history in the above example, but the configuration is not limited thereto. Clinical data related to one patient may be set as a processing unit. That is, a period from when a user starts viewing clinical data of a specific patient to when the viewing of the clinical data of that patient ends may be set as a processing unit.

Above are descriptions of the medical information processing apparatus 1 according to the present embodiment. The medical information processing apparatus 1 specifies clinical data associated with clinical data being viewed by a user based on a viewing history of the user and then collectively displays the clinical data of the two as a list or a graph. Thereby, the medical information processing apparatus 1 can reduce or resolve a burden on a user of changing the layout of the display screen, such as manually adding a panel of clinical data that the user wants to view. Furthermore, since each piece of clinical data that the user wants to view is gathered on one collectively displayed graph, the user can, for example, easily compare each piece of clinical data, i.e., view the clinical data efficiently.

According to at least one embodiment described above, it is possible to improve the convenience of viewing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising processing circuitry configured to:
designate one or more pieces of first clinical data among a plurality of pieces of clinical data viewed by a user;
specify one or more pieces of second clinical data associated with the first clinical data among the plurality of pieces of clinical data based on a viewing history related to the plurality of pieces of clinical data; and
output the first clinical data and the second clinical data.

2. The medical information processing apparatus according to claim 1, wherein
when the first clinical data and the second clinical data are time-series data,
the processing circuitry is configured to designate a first time zone on a time axis of the first clinical data, and specify the second clinical data in a second time zone that coincides or approximately coincides with the first time zone on a time axis of the second clinical data.

3. The medical information processing apparatus according to claim 1, wherein
the viewing history is each combination of the plurality of pieces of clinical data viewed simultaneously by the user, and
the processing circuitry is configured to specify one or more pieces of clinical data that are included in one or more first combinations including the first clinical data and are different from the first clinical data, as the second clinical data.

4. The medical information processing apparatus according to claim 3, wherein
the viewing history is a viewing time or a viewing frequency for the each combination of the plurality of pieces of clinical data viewed simultaneously by the user, and
the processing circuitry is configured to specify one or more second combinations including a viewing time or a viewing frequency that is equal to or greater than a threshold value, among the first combinations, and then specify one or more pieces of clinical data that are included in the second combinations and are different from the first clinical data, as the second clinical data.

5. The medical information processing apparatus according to claim 1, wherein
the viewing history is a viewing order of the plurality of pieces of clinical data viewed by the user, and
the processing circuitry is configured to specify, as the second clinical data, one or more pieces of clinical data viewed within ±Nth (N is a natural number) from the first clinical data as a standard.

6. The medical information processing apparatus according to claim 1, wherein the processing circuitry is configured to output display data for displaying the second clinical data in association with the first clinical data as a pop-up or display data for collectively displaying the first clinical data and the second clinical data on a common time axis.

7. The medical information processing apparatus according to claim 6, wherein the processing circuitry is configured to associate a comment input by the user with the display data.

8. The medical information processing apparatus according to claim 7, wherein the processing circuitry is configured to transmit the display data and the comment to one or more other users different from the user.

9. A medical information processing system comprising a medical information processing apparatus and a terminal, wherein
the medical information processing apparatus includes processing circuitry configured to:
designate one or more pieces of first clinical data among a plurality of pieces of clinical data viewed by a user who operates the terminal;
specify one or more pieces of second clinical data associated with the first clinical data among the plurality of pieces of clinical data based on a viewing history related to the plurality of pieces of clinical data; and
output the first clinical data and the second clinical data to the terminal, and
the terminal includes processing circuitry configured to display the first clinical data and the second clinical data.
